(19) United States
(12) Patent Application Publication
(10) Pub. No.: US 2024/0374754 A1
(43) Pub. Date: Nov. 14, 2024

(54) MRNA DELIVERY USING LIPID NANOPARTICLES

(71) Applicant: THE UNIVERSITY OF BRITISH COLUMBIA, Vancouver (CA)

(72) Inventors: Jayesh A. KULKARNI, Vancouver (CA); Nisha CHANDER, Vancouver (CA); Genc BASHA, Vancouver (CA); Marco A. CIUFOLINI, Vancouver (CA); Dominik WITZIGMANN, Binzen (DE); Pieter R. CULLIS, Vancouver (CA)

(73) Assignee: THE UNIVERSITY OF BRITISH COLUMBIA, Vancouver, BC (CA)

(21) Appl. No.: 18/564,588

(22) PCT Filed: May 31, 2022
(86) PCT No.: PCT/CA2022/050868

§ 371 Date: Nov. 30, 2023

Related U.S. Application Data
(60) Provisional application No. 63/195,269, filed on Jun. 1, 2021.

(51) Int. Cl. A61K 48/00; A61K 9/51; C12N 15/88

(52) U.S. Cl. CPC A61K 48/0041; A61K 9/5123; C12N 15/88

(57) ABSTRACT

The present disclosure provides a lipid nanoparticle comprising encapsulated mRNA and at least 30 mol % of a sphingolipid (e.g. sphingomyelin (SM)), and at least one of a sterol and a hydrophilic polymer-lipid conjugate, the lipid nanoparticle comprising a core comprising an electron dense region and an aqueous portion surrounded at least partially by a lipid layer comprising a bilayer and the lipid nanoparticle exhibiting at least a 2-fold increase in gene expression in the liver, spleen and/or bone marrow at 4 or 24 hours post-injection as compared to a lipid nanoparticle encapsulating mRNA with an Onpattro™-type formulation of cationic, ionizable lipid/DSPC or ESM/cholesterol/PEG-lipid at 50/10/38.5/1.5, mol:mol, wherein the gene expression is measured in an animal model by detection of green fluorescent protein (GFP). Further provided are methods of medical treatment and uses of such lipid nanoparticles to treat or prevent a disease condition in a hepatic or non-hepatic tissue or organ.

### CROSS REFERENCE TO RELATED APPLICATION

This application claims the benefit of U.S. Provisional Patent Application Ser. No. 63/195,269 filed 1 Jun. 2021, entitled “MRNA DELIVERY USING LIPID NANOPARTICLES”.

### TECHNICAL FIELD

The present disclosure relates to a lipid nanoparticle formulation for delivery of mRNA.

### BACKGROUND

Lipid nanoparticle (LNP) formulations represent a revolution in the field of nucleic acid delivery. An early example of a lipid nanoparticle product approved for clinical use is Onpattro™ developed by Alnylam. Onpattro™ is a lipid nanoparticle-based short interfering RNA (siRNA) drug for the treatment of polyneuropathies induced by hereditary transthyretin amyloidosis.

The Onpattro™ LNP formulation consists of four main lipid components: ionizable amino lipid, distearoylphosphatidylcholine (DSPC), cholesterol, and polyethylene glycol conjugated lipids (PEG-lipids) at respective molar amounts of 50/10/38.5/1.5. Onpattro™ is still considered the gold standard for comparison in studies of LNP-mediated efficacy and current approaches to LNP design make few deviations from the four-component system.

Of these four components, the ionizable lipid is considered important for the in vivo potency of the LNP system. Accordingly, most work in the field has focused primarily on improving this lipid component. The ionizable lipid is typically positively charged at low pH, which facilitates association with the negatively charged nucleic acid but is neutral at physiological pH, making it more biocompatible in biological systems. Further, it has been suggested that after the lipid nanoparticles are taken up by a cell by endocytosis, the ability of these lipids to ionize at low pH enables endosomal escape. This in turn allows the nucleic acid to be released into the intracellular compartment where it can exert its therapeutic effect.

With respect to the remaining three lipid components, the PEG-lipid is well known for preventing aggregation of the LNP and cholesterol functions to stabilize the particle. The DSPC is a bilayer-forming lipid, and provides a structural role in the LNP membrane.

The success of the four-component Onpattro™ LNP delivery system led to the clinical development of the leading LNP-based COVID-19 mRNA vaccines. Since mRNA rapidly degrades in the body, lipid nanoparticles are used to encapsulate mRNA and reduce such degradation. Indeed, the recent covid19 Pfizer/BioNTech vaccine relies on lipid nanoparticles to deliver mRNA to the cytoplasm of host cells. In the case of the covid19 vaccine, the mRNA encodes the Sars-Cov-2 spike protein. However, messenger RNA (mRNA) LNP therapy has potential to treat diseases beyond covid19. Such therapy could be more broadly applicable to any disease or condition that can be treated or prevented by the production of a protein or peptide encoded by the mRNA. Thus, there is tremendous potential for mRNA to treat human disease. Most work on LNP mRNA systems for intravenous administration has investigated gene expression in the liver. Similar to siRNA carrier systems, the focus of research efforts has been primarily on developing improved ionizable cationic lipids within an “Onpattro™” lipid composition (ionizable lipid/DSPC/cholesterol/PEG-lipid; 50/10/38.5/1.5 mol:mol).^{[11]}.

However, there is an on-going need to develop more effective biocompatible and transfection competent LNPs for mRNA delivery. Such LNPs most advantageously will display enhanced in vivo gene expression to a target cell, organ or tissue relative to known formulations.

The present disclosure seeks to address one or more of these ongoing needs and/or provide useful alternatives to mRNA formulations over those described in the art.

### SUMMARY

In some embodiments, the lipid nanoparticles (LNPs) described herein may exhibit an unusual morphology as revealed by cryo-TEM with a core having an electron dense region and an aqueous portion partially or completely surrounded by a lipid layer comprising at least a bilayer. By virtue of such unique characteristics, the mRNA LNPs prepared in accordance with embodiments of the disclosure may be especially suitable for enhanced gene expression in one or more target cells, tissues or organs, thereby expanding the clinical utility of mRNA therapeutics.

In further embodiments, the present disclosure is based, in part, on the finding that LNPs for the delivery of mRNA formulated with elevated levels of sphingomyelin (SM) may exhibit high mRNA trapping efficiencies, such as greater than 90% in some embodiments. Surprisingly, these LNPs may exhibit mRNA transfection potencies in vitro that are comparable or superior to those observed for LNP mRNA systems with the “Onpattro-type” lipid compositions described herein. In further examples of the disclosure, LNPs comprising elevated levels of sphingomyelin exhibit significantly improved in vivo translation of mRNA in a target organ or tissue relative to an Onpattro-type formulation. In some embodiments, the LNPs herein may exhibit improved translation of mRNA in hepatocytes over splenocytes and bone marrow cells.

According to one aspect of the disclosure, there is provided a lipid nanoparticle comprising encapsulated mRNA and 30 to 60 mol % of sphingolipid, and at least one of a sterol and a hydrophilic polymer-lipid conjugate, the lipid nanoparticle comprising a core having an electron dense region and an aqueous portion surrounded at least partially by a lipid layer comprising at least a bilayer and the lipid nanoparticle exhibiting at least a 2-fold increase in gene expression in the liver, spleen and/or bone marrow at 4 or 24 hours post-injection as compared to a lipid nanoparticle encapsulating the mRNA with a formulation of ionizable lipid/DSPC/cholesterol/PEG-lipid at 50/10/38.5/1.5 or ionizable lipid/egg sphingomyelin (ESM)/cholesterol/PEG-lipid, mol:mol, wherein the gene expression is measured in an animal model by detection of green fluorescent protein (GFP). Alternatively, the gene expression is measured by detection of luciferase (e.g., see Example 6).

According to another aspect of the disclosure, there is provided a lipid nanoparticle for hepatic or extrahepatic delivery of mRNA, the lipid nanoparticle comprising: (i) encapsulated mRNA; (ii) a sphingolipid lipid content of from 30 to 60 mol % of total lipid present in the lipid nanoparticle; (iii) a cationic lipid content of from 5 mol % to 50 mol % of the total lipid; (iv) a sterol selected from cholesterol or a derivative thereof; and (v) a hydrophilic polymer-lipid conjugate that is present at 0.5 mol % to 5 mol % of the total lipid.

In certain embodiments of either of the foregoing aspects, the lipid nanoparticle is visualized by cryo-EM microscopy, wherein the lipid nanoparticles contain an electron dense region either (i) enveloped by the aqueous portion, or (ii) partially surrounded by the aqueous portion and

wherein a portion of a periphery of the electron dense region is contiguous with the lipid layer comprising at least a bilayer. In some embodiments, at least a portion of the mRNA is encapsulated in the electron dense region or the lipid bilayer.

In some embodiments, the sphingolipid content is between 30 mol % and 50 mol %.

In alternative embodiments, the sphingolipid content is between 35 mol % and 60 mol %.

In a further embodiment, the cationic lipid is an ionizable lipid. An example of a suitable cationic lipid is an amino lipid.

In some embodiments, the hydrophilic polymer-lipid conjugate is a polyethyleneglycol-lipid conjugate.

In further embodiments, the sterol is present at from 15 mol % to 50 mol % based on the total lipid present in the lipid nanoparticle.

In further embodiments, the sterol is present at from 18 mol % to 45 mol % based on the total lipid present in the lipid nanoparticle.

In another embodiment, the sphingolipid is a sphingomyelin.

In a further embodiment, the mRNA stability of the lipid nanoparticle is improved relative to the formulation of ionizable, cationic lipid/DSPC/cholesterol/PEG-lipid at 50/10/38.5/1.5 or cationic lipid/sphingomyelin/cholesterol/PEG-lipid at 50/10/38.5/1.5, mol:mol as measured by quantifying degradation in an in vito assay by determining band intensity using a denaturing agarose gel after incubation of the lipid nanoparticle with fetal bovine serum for 2, 4 or 24 hours.

In another aspect, there is provided a method for in vivo delivery of mRNA to a hepatic or extrahepatic tissue or organ to treat or prevent a disease or disorder in a mammalian subject, the method comprising: administering to the mammalian subject a lipid nanoparticle according to any one of the aspects and/or embodiments described above.

In a further embodiment, the lipid nanoparticle is for delivery to spleen, bone marrow and/or liver.

The disease or disorder may be a viral infection or a cancer.

The disclosure also provides a use of the lipid nanoparticle as described in any one of the foregoing aspects or embodiments for in vivo delivery of mRNA to spleen, bone marrow or liver to treat or prevent a disease or disorder in a mammalian subject.

In one embodiment, the use is to treat or prevent a disease or disorder of an extrahepatic tissue or organ. In another embodiment, the disease or disorder is a viral infection or cancer.

A further aspect provides use of the lipid nanoparticle according to any one of the above aspects or embodiments for the manufacture of a medicament for in vivo delivery of mRNA to a hepatic or extrahepatic tissue or organ to treat or prevent a disease or disorder in a mammalian subject.

### BRIEF DESCRIPTION OF THE DRAWINGS

FIG. 1 shows an in vitro screen to analyze the effect of different helper lipids on transfection of LNP formulations with Luciferase mRNA in HuH7 cells. HuH7 cells were treated with a variety of LNPs containing ESM at 40 mol % of helper lipids with luciferase mRNA over a dose range of 0.1-3 μg/mL for up to 24 hours, following which luciferase expression was quantified using luminescence. Formulations containing 40 mol % ESM showed over a 7-fold increase in luciferase expression at a 3 μg/mL mRNA concentration compared to the 10 mol % ESM or DSPC control formulation.

FIG. 2 shows representative cryo-TEM images of LNPs containing increasing proportions of ESM. For complete details of lipid composition see Table 1. A: 10 mol % ESM; B: 40 mol % ESM.

FIG. 3 shows the experimental design for in vivo studies examining the potency of LNP-GFP mRNA formulations. A: Mice (n=2) were intravenously injected with phosphate buffered saline (PBS); an Onpattro™-type (ESM-10) LNP; or ESM-40 formulations. For complete details of lipid composition see Table 2, B: shows the organs examined for GFP expression.

FIG. 4 shows in vivo potency of LNP-GFP mRNA formulations in the liver of C57B16 mice. A-C: Flow cytometry data indicating uptake of LNPs assessed by gating the target cell populations (ASGPR1-PE-Cy7) in the liver expressing the DiD above the background levels 4 hours post-injection (hpi), D: GFP translation determined by gating the cells co-expressing DiD and GFP, E: GFP expression levels in cells co-expressing DiD and GFP 4 hpi.

FIG. 5 shows in vivo potency of LNP-GFP mRNA formulations in the liver of C57B16 mice. A-C: Flow cytometry data indicating uptake of LNPs assessed by gating the target cell populations (ASGPR1-PE-Cy7) in the liver expressing the DiD above the background levels 24 hpi, D: GFP translation determined by gating the cells co-expressing DiD and GFP, E: GFP expression levels in cells co-expressing DiD and GFP 24 hpi.

FIG. 6 shows in vivo potency of LNP-GFP mRNA formulations in the spleen of C57B16 mice. A-C: Flow cytometry data indicating uptake of LNPs assessed by gating the target cell populations (ASGPR1-PE-Cy7) in the liver expressing the DiD above the background levels 4 hpi, D: GFP translation determined by gating the cells co-expressing DiD and GFP, E: GFP expression levels in cells co-expressing DiD and GFP 4 hpi.

FIG. 7 shows in vivo potency of LNP-GFP mRNA formulations in the spleen of C57B16 mice. A-C: Flow cytometry data indicating uptake of LNPs assessed by gating the target cell populations (ASGPR1-PE-Cy7) in the liver expressing the DiD above the background levels 24 hpi, D: GFP translation determined by gating the cells co-expressing DiD and GFP, E: GFP expression levels in cells co-expressing DiD and GFP 24 hpi.

FIG. 8 shows in vivo potency of LNP-GFP mRNA formulations in the bone marrow of C57B16 mice. A-C: Flow cytometry data indicating uptake of LNPs assessed by gating the target cell populations (ASGPR1-PE-Cy7) in the liver expressing the DiD above the background levels 4 hpi, D: GFP translation determined by gating the cells co-expressing DiD and GFP, E: GFP expression levels in cells co-expressing DiD and GFP 4 hpi.

FIG. 9 shows in vivo potency of LNP-GFP mRNA formulations in the bone marrow of C57B16 mice. A-C: Flow cytometry data indicating uptake of LNPs assessed by gating the target cell populations (ASGPR1-PE-Cy7) in the liver expressing the DiD above the background levels 24 hpi, D: GFP translation determined by gating the cells co-expressing DiD and GFP, E: GFP expression levels in cells co-expressing DiD and GFP 24 hpi.

FIG. 10 shows the ESM40 formulation increases levels of mRNA translation into the encoded protein relative to UBC005 (DSPC10) formulation at 5 hours post-administration. LNP-Luc mRNA under different formulations was administrated to Female CD1 mice intravenously (2 mg/kg) and D-luciferin (150 mg/kg) was administrated intraperitoneally. A: Ex vivo bioluminescence images of 15 organs (brain, thymus, heart, lungs, liver, spleen, pancreas, intestine, kidney, muscle, and bone) using an IVIS Spectrum imaging system. B: Quantified bioluminescence values the region of interest measured by photon flux (photons/second) using the Living IMAGE Software. For complete details of lipid composition see Table 3.

FIG. 11 shows the ESM40 formulation increase levels of mRNA translation into the encoded protein relative to UBC005 (DSPC10) formulation at 24 hours post-administration. LNP-Luc mRNA under different formulations was administrated to Female CD1 mice intravenously (2 mg/kg) and D-luciferin (150 mg/kg) was administrated intraperitoneally. A: Ex vivo bioluminescence images of 15 organs (brain, thymus, heart, lungs, liver, spleen, pancreas, intestine, kidney, muscle, and bone) using an IVIS Spectrum imaging system. B: Quantified bioluminescence values the region of interest measured by photon flux (photons/second) using the Living IMAGE Software. For complete details of lipid composition see Table 3.

FIG. 12 shows that an ESM40 formulation comprising 40 mol % ESM (ESM40) improves mRNA stability in serum at 37° C. over 24 hours relative to a UBC005 (DSPC10) formulation. A: Normalized absorption ratio between 260 nm/280 nm to access mRNA degradation. B: RNA 1% denaturing agarose gel stained with gel red loaded with 120 ng RNA extracted from LNP-mRNA. C: Electropherogram of Agilent Bioanalyzer 2100 analysis of the extracted mRNA samples incubated in 50% FBS or PBS. For complete details of lipid composition see Table 3.

Other objects, features, and advantages of the present invention will be apparent to one of skill in the art from the following detailed description and figures.

### DETAILED DESCRIPTION

Encapsulated mRNA

The lipid nanoparticle described herein comprises encapsulated mRNA. As used herein, the term “messenger RNA (mRNA)” refers to a polynucleotide that encodes at least one peptide, polypeptide or protein. The term is meant to include, but is not limited to, small activating RNA (saRNA) and transamplifying RNA (taRNA).

As used herein, the term “encapsulation,” with reference to incorporating the mRNA molecule within a nanoparticle refers to any association of the mRNA with any component or compartment of the lipid nanoparticle.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is chemically synthesized molecules, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before formulation and encapsulation to remove undesirable impurities including various enzymes and other reagents used during mRNA synthesis.

The present disclosure may be used to formulate and encapsulate mRNAs of a variety of lengths. In some embodiments, the present disclosure may be used to formulate and encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

While mRNA provided from in vitro transcription reactions may be desirable in certain embodiments, other sources of mRNA are contemplated, such as mRNA produced from bacteria, fungi, plants, and/or animals.

### Helper Lipid

In the context of the present disclosure, the term “helper lipid” includes a lipid selected from sphingomyelin, or mixtures thereof, such as a mixture of a sphingolipid, such as sphingomyelin and a phosphatidycholine lipid.

By the term “sphingolipid”, it is mean a class of lipids comprising a backbone of sphingoid bases that are suitable for formulation in the LNPs herein and includes sphingomyelin. The sphingomyelin may have a phosphocholine, ceramide or phosphoethanolamine head group.

In some embodiments, the helper lipid is selected from sphingomyelin, or mixtures of sphingomyelin and distearoylphosphatidylcholine (DSPC), dioleoylphosphatidylcholine (DOPC), 1-palmitoyl-2-oleoyl-phosphatidylcholine (POPC) and dipalmitoyl-phosphatidylcholine (DPPC). In certain embodiments, the helper lipid is egg sphingomyelin (ESM) or is synthesized using known synthetic techniques.

The helper lipid content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of helper lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the helper lipid content is from 20 mol % to 60 mol % or 25 mol % to 60 mol % or 30 mol % to 60 mol % or 35 mol % to 60 mol % or 40 mol % to 60 mol % of total lipid present in the lipid nanoparticle.

The sphingomyelin content of the lipid nanoparticle in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of sphingomyelin content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the sphingomyelin content is from 20 mol % to 60 mol % or 25 mol % to 60 mol % or 30 mol % to 60 mol % or 35 mol % to 60 mol % or 40 mol % to 60 mol % of total lipid present in the lipid nanoparticle.

The helper lipid content is determined based on the total amount of lipid in the lipid nanoparticle, including the sterol.

### Cationic Lipid

The term “cationic lipid” refers to any of a number of lipid species that carry a net positive charge at a selected pH. It should be understood that a wide variety of ionizable lipids can be used in the practice of the disclosure. For example, the cationic lipid may be an ionizable lipid that has a pKa such that the lipid is substantially neutral at physiological pH (e.g., pH of about 7.0) and substantially charged at a pH below its pKa. The pKa of the ionizable lipid may be less than 7.5, or more typically less than 7.0. In some embodiments, the cationic lipid has a head group comprising an amino group. In some cases, the cationic lipids comprise a protonatable tertiary amine (e.g., pH titratable) head group, C16 to C18 alkyl chains, a linker region between the head group and alkyl chains, and 0 to 3 double bonds in the alkyl chains. Optionally, the alkyl chains are branched. Such lipids include but are not limited to lipids having sulfur atoms in their lipophilic tails, such as MF019 or other sulfur lipids described in Formula I of commonly owned PCT/CA2022/050042 filed on Jan. 12, 2022, which is incorporated herein by reference.

It will be appreciated that the foregoing ionizable lipids are merely illustrative of exemplary embodiments. For example, the cationic lipids may have biodegradable groups in their lipophilic chains and/or branched chains, among other modifications.

The cationic lipid content may be less than 60 mol %, less than 55 mol %, less than 50 mol, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the cationic lipid content is from 5 mol % to 60 mol % or 10 mol % to 55 mol % or 10 mol % to 50 mol % or 15 mol % to 45 mol % or 20 mol % to 40 mol % of total lipid present in the lipid nanoparticle.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative. Examples of derivatives include β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β[N—(N′N′-dimethylaminoethyl) carbamoyl cholesterol (DC-Chol), 24 (S)-hydroxycholesterol, 25-hydroxycholesterol, 25 (R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) helper lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the particle. The conjugate includes a vesicle-forming lipid having a polar head group, and (ii) covalently attached to the head group, a polymer chain that is hydrophilic. Example of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (G_{M1}). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In certain embodiments, the hydrophilic polymer lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

### Nanoparticle Preparation and Morphology

Delivery vehicles incorporating the mRNA and having a core comprising an electron dense region and an aqueous portion surrounded at least partially by a lipid layer (e.g., comprising at least a bilayer) can be prepared using a variety of suitable methods, such as a rapid mixing/ethanol dilution process. Examples of preparation methods are described in Jeffs, L. B., et al., Pharm Res, 2005, 22 (3): 362-72; and Leung, A. K., et al., The Journal of Physical Chemistry. C, Nanomaterials and Interfaces, 2012, 116 (34): 18440-18450, each of which is incorporated herein by reference in its entirety.

Without being bound by theory, the mechanism whereby a lipid nanoparticle comprising encapsulated mRNA can be formed using the rapid mixing/ethanol dilution process can be hypothesized as beginning with formation of a dense region of hydrophobic mRNA-ionizable lipid core at pH 4 surrounded by a monolayer of helper lipid/cholesterol that fuses with smaller empty vesicles as the pH is raised due to the conversion of the ionizable cationic lipid to the neutral form.^{[29,30]} As the proportion of bilayer helper lipid increases, the bilayer lipid progressively forms blebs and the ionizable lipid migrates to the interior hydrophobic core. At high enough helper lipid contents, the exterior bilayer preferring helper lipid can form a complete bilayer around the interior trapped volume.

By the term “core”, it is meant a trapped volume of the nanoparticle that comprises an aqueous portion and an electron dense region. The aqueous portion and electron dense region can be visualized by cryo-EM microscopy. The electron dense region within the core is either only partially surrounded by the aqueous portion within the enclosed space or optionally entirely surrounded or enveloped by the aqueous portion within the core. For example, a portion of a periphery of the electron dense region within the core may be contiguous with the lipid layer of the lipid nanoparticle. In one embodiment, qualitatively, generally around 10-70% or 10-50% of the periphery of the electron dense region may be visualized as contiguous with a portion of the lipid layer of the lipid nanoparticle by cryo-EM microscopy.

In one embodiment, the electron dense region is generally spherical in shape. In another embodiment, the electron dense region is hydrophobic.

The lipid nanoparticles herein may exhibit particularly high trapping efficiencies of mRNA. Thus, in one embodiment, the trapping efficiency is at least 70, 75, 80, 85 or 90%.

In one embodiment, the mRNA is at least partially encapsulated in the electron dense region. For example, in one embodiment, at least 50, 60, 70 or 80 mol % of the mRNA is encapsulated in the electron dense region. In another embodiment, at least 50, 60, 70 or 80 mol % of the ionizable lipid is in the electron dense region.

The lipid nanoparticle may comprise a single bilayer or comprise multiple concentric lipid layers (i.e., multi-lamellar). The one or more lipid layers, including the bilayer, may form a continuous layer surrounding the core or may be discontinuous. The lipid layer may be a combination of a bilayer and a monolayer in some embodiments. In one embodiment, the lipid layer is a continuous bilayer that surrounds the core.

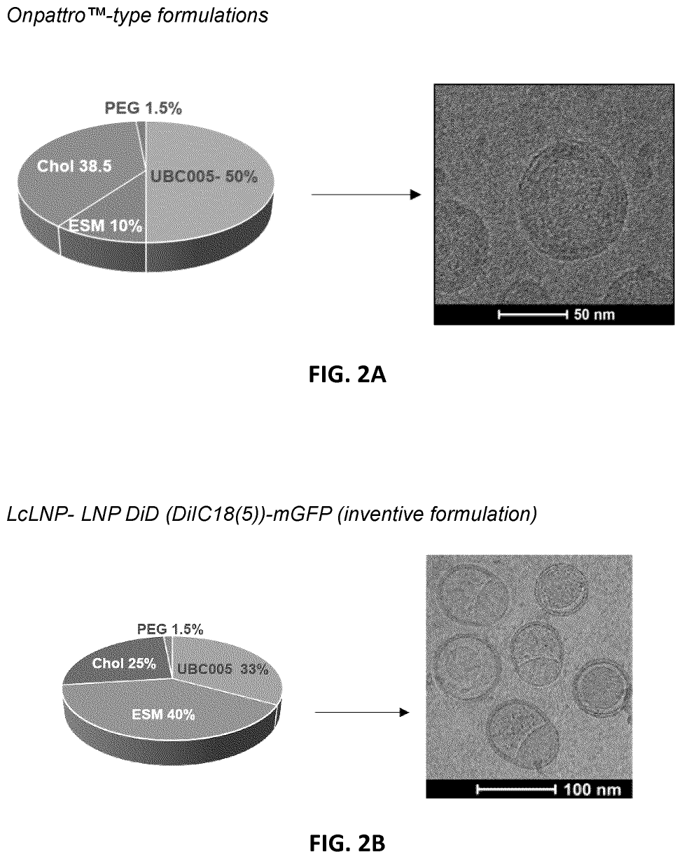

The lipid nanoparticle of the present disclosure possesses a unique morphology as visualized by cryo-EM (see FIG. 2B). In one non-limiting example, as the helper lipid content increases, the core assumes a morphology in which the electron dense region is surrounded and “floats” within the aqueous portion, which in turn is surrounded by the lipid bilayer. (Compare FIG. 2A and FIG. 2B).

In one particularly advantageous embodiment, the unique morphology of the LNP at high sphingolipid content enables stable encapsulation of the mRNA. In particular, the bilayer surrounding the core may improve in vivo stability of the lipid nanoparticle after administration. Such a bilayer is not observed in formulations of 10 mol % helper lipid (e.g., formulations of 50/10/38.5/1.5 cationic, ionizable lipid/helper lipid/cholesterol/PEG-lipid mol: mol, wherein the helper lipid is DSPC or ESM). Without being limited by theory, the bilayer may protect the mRNA encapsulated within the core from in vivo degradation. Consequently, the LNP of the disclosure may provide significant improvements in delivery of mRNA to a target site.

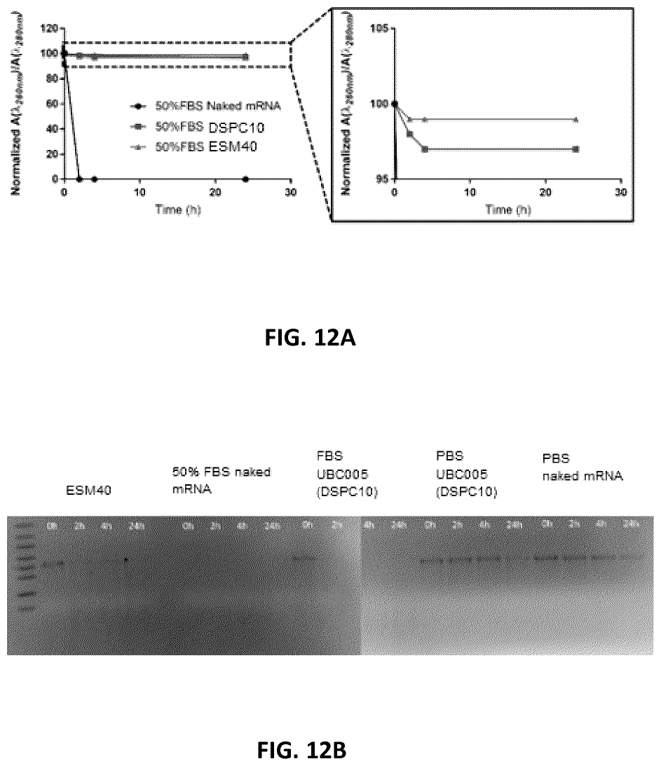

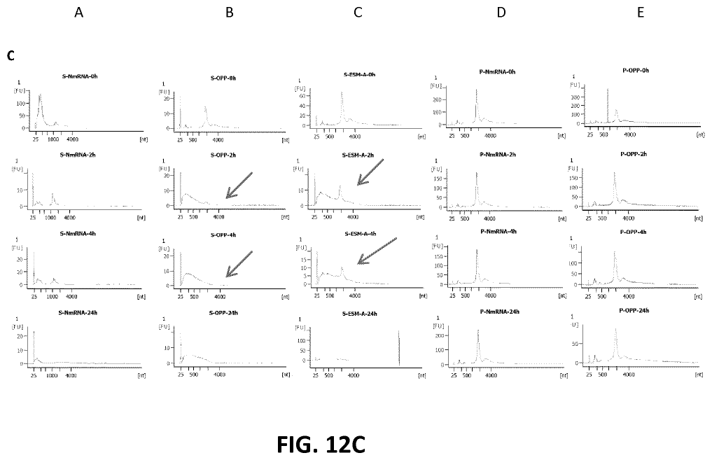

In one embodiment, the stability of the mRNA encapsulated in the lipid nanoparticle having elevated sphingolipid (e.g., sphingomyelin) is improved as determined by quantifying mRNA degradation in an in vitro assay. The LNP samples tested are incubated with fetal bovine serum (FBS) for a defined time period and subsequently the samples are run on an agarose gel to determine band intensity of mRNA extracted from the LNP. The duration of incubation of LNPs and appropriate controls with serum may be conducted for 0, 2, 4 and 24 hours. The agarose gel is a denaturing gel and mRNA may be quantified by light absorption of the bands. The band intensity may be measured by absorption at appropriate wavelengths and in some embodiments, the normalized absorption ratio for peaks at λ 260 nm and λ 280 nm (λ 260 nm/λ 280 nm) for the formulation of the invention is at least 0.5, 1.0, 1.5 or 2% greater than that of a control formulation having the UBC005 DSPC-10 formulation at one or more of 2, 4 or 24 hours post-incubation in fetal bovine serum (e.g., see Example 7 and FIG. 12A). The procedure for measuring mRNA stability is set out in Example 7.

Thus, in certain embodiments the electron dense region of the core is separated from the lipid layer comprising the bilayer by the aqueous portion. For example, the disclosure provides a lipid nanoparticle preparation comprising a plurality of lipid nanoparticles in which at least 20%, 30%, 40%, 50%, 60% or 70% of the particles as determined by cryo-EM microscopy have a core having an electron dense region that is surrounded by the aqueous portion and in which the aqueous portion is surrounded by the lipid layer comprising the bilayer as visualized by cryo-EM microscopy.

In another embodiment, and without being limiting, the disclosure provides a lipid nanoparticle preparation comprising a plurality of lipid nanoparticles in which generally at least 20%, 30%, 40%, 50%, 60% or 70% of the particles have an electron dense region of the core surrounded or enveloped by a continuous aqueous space disposed between the lipid layer and the aqueous portion as visualized by cryo-EM microscopy.

The average particle size of a preparation of the lipid nanoparticles may be between 40 and 120 nm or between 45 and 115 nm.

Improved Gene Expression in Liver, Spleen and/or Bone Marrow at 4 or 24 Hours

As used herein, “expression” of an mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme).

The unique morphology of the lipid nanoparticle may facilitate long circulation lifetimes thereof after administration to a patient, thereby improving mRNA delivery to a wider range of tissues than previous formulations for mRNA delivery, including but not limited to delivery to the liver, spleen and/or bone marrow. Whether or not a lipid particle exhibits such enhanced delivery to a given tissue or organ can be determined by biodistribution studies an in vivo mouse model. In such embodiments, green fluorescent protein (GFP) may be used to detect mRNA expression in a given tissue or organ. In particular, according to such embodiments, LNP mRNA systems are prepared using mRNA coding for GFP and biodistribution and GFP expression evaluated using flow cytometry following systemic administration.

To assess whether a given lipid nanoparticle exhibits an increase in gene expression in a relevant tissue or organ at 4 or 24 hours post-injection, the two formulations being compared are identical apart from the content of helper lipid and are subjected to the same experimental methods and materials to determine in vivo expression. Expression is measured as set forth in Example 3.

In one embodiment, the lipid nanoparticle exhibits at least a 10%, 20%, 30%, 40%, 50%, 60%, 70%, 80%, 90%, 100%, 110%, 120%, 130%, 140%, 150%, 160%, 170%, 180%, 190% or 200% increase in gene expression in vivo in liver, spleen and/or bone marrow at 4 or 24 h post-injection as compared to a lipid nanoparticle encapsulating mRNA with an “Onpattro-type” formulation of ionizable lipid/DSPC/cholesterol/PEG-lipid at 50/10/38.5/1.5; mol: mol, wherein the gene expression is measured in an animal model by detection of green fluorescent protein (GFP). In one embodiment, the above levels of increased gene expression are observed in the liver.

In one embodiment, the lipid nanoparticle exhibits at least a 2-fold, 3-fold, 4-fold, 5-fold, 6-fold, 7-fold, 8-fold, 9-fold, 10-fold, 11-fold or 12-fold increase in gene expression in vivo in the liver, spleen and/or bone marrow at 4 or 24 h post-injection as compared to a lipid nanoparticle encapsulating mRNA with an “Onpattro-type” formulation of ionizable lipid/DSPC/cholesterol/PEG-lipid at 50/10/38.5/1.5; mol: mol, wherein the gene expression is measured in an animal model by detection of GFP. The upper limit of gene expression may be 30-fold, 25-fold or 20-fold increase in gene expression in vivo in the liver at 4 or 24 h post-injection as compared to a lipid nanoparticle encapsulating mRNA with the “Onpattro-type” formulation.

### Pharmaceutical Formulations

In some embodiments, the lipid nanoparticle comprising mRNA is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical composition is administered parenterally, i.e., intra-arterially, intravenously, subcutaneously or intramuscularly. In yet a further embodiment, the pharmaceutical compositions are for intra-tumoral administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject. The examples are intended to illustrate the preparation of specific lipid nanoparticle mRNA preparations and properties thereof but are in no way intended to limit the scope of the invention.

### EXAMPLES

### Materials and Methods

### Materials

The lipids 1,2-distearoyl-sn-glycero-3-phosphorylcholine (DSPC) and N-(hexadecanoyl)-sphing-4-enine-1-phosphocholine (ESM) were purchased from Avanti Polar Lipids (Alabaster, AL). The ionizable lipid used was ((6Z,9Z,26Z,29Z)-Pentatriaconta-6,9,26,29-tetraen-18-yl 4-(dimethylamino) butanoate (chemical formula C₄₁H₇₅NO₂; molecular weight 614.06); also referred to within the Examples below as “ionizable, cationic lipid” or “UBC005”. Cholesterol, sodium acetate, Dulbecco's phosphate buffered saline (PBS), fetal bovine serum (FBS), and Triton X-100 were purchased from Sigma-Aldrich (St. Louis, MO). (R)-2,3-bis(octadecyloxy) propyl-1-(methoxy polyethylene glycol 2000) carbamate (PEG-DMG) was provided by Alnylam Pharmaceuticals. Lipid tracers 1,1′-dioctadecyl-3,3,3′,3′-tetramethylindocarbocyanine perchlorate (DiI-C18) and 1,1′-Dioctadecyl-3,3,3′,3′-Tetramethylindodicarbocyanine, 4-Chlorobenzenesulfonate Salt (DiD-C18) were purchased from Invitrogen (Burlington, ON). Dulbecco's Modified Eagle Medium (DMEM) was purchased from ThermoFischer Scientific. Luciferase mRNA was provided by Dr. Drew Weismann's lab (University of Pennsylvania). CleanCap® EGFP mRNA (5moU) was purchased from TriLink Biotechnologies (San Diego, CA).

### Methods

Lipid Nanoparticle-mRNA preparation using T-tube mixing: The ionizable, cationic lipid (see Materials above), helper lipid (DSPC or ESM), cholesterol and PEG-DMG were dissolved in ethanol at varying mole ratios. The lipids in ethanol and mRNA prepared in 25 mM acetate buffer (pH 4.0) were combined using the T-tube formulation method at total flow rate of 20 mL/min and flow rate ratio of 3:1 aqueous: organic phases (v/v). Following formulation, particles were dialyzed against Dulbecco's phosphate buffered saline (PBS) (pH 7.4) using 12-14 kDa regenerated cellulose membranes (Spectrum Labs, Rancho Dominguez, 38 CA) overnight to remove residual EtOH.

Analysis of Lipid Nanoparticles size and morphology: LNP size and morphology were determined using cryogenic-transmission electron microscopy (cryoTEM) as described previously.^{[16.17]} LNP size (number weighting) and polydispersity indexes (PdI) was further confirmed by dynamic light scattering (DLS) using the Malvern Zetasizer NanoZS (Worcestershire, UK). Total lipid was determined by measuring the cholesterol content using the Cholesterol E assay (Wako Chemicals, Richmond, VA) at an absorbance of 260 nm.

Analysis of mRNA encapsulation efficiency: mRNA encapsulation efficiency was determined using the Quant-iT Ribogreen RNA assay (Life Technologies, Burlington, ON). Briefly, LNP-mRNA was incubated at 37° C. for 10 min in the presence or absence of 1% Triton X-100 (Sigma-Aldrich, St. Louis, MO) followed by the addition of the ribogreen reagent. The fluorescence intensity (Ex/Em: 480/520 nm) was determined and samples treated with Triton X-100 represent total mRNA while untreated samples represent unencapsulated mRNA.

Cryogenic transmission electron microscopy (Cryo-TEM): LNPs loaded with mRNA were concentrated (Amicon Ultra-15 Centrifuge Filter Units, Millipore, Billerica, MA) to a total lipid concentration of ˜25 mg/mL prior to analysis. Formulations were deposited onto glow-discharged copper grids and vitrified using a FEI Mark IV Vitrobot (FEI, Hillsboro, OR). Cryo-TEM imaging was performed using a 200 kV Glacios microscope equipped with a Falcon III camera at the UBC High Resolution Macromolecular Cryo-Electron Microscopy facility (Vancouver, BC).

In vitro gene expression assay for mRNA-LNPs: Luciferase gene expression was performed using HuH7 cells-hepatocyte derived carcinoma cell line. Growth media was composed of DMEM with FBS (10%). Cells were plated in 96-well cell culture treated plates (Falcon/Corning Inc., Corning, NY) at a density of 12,500 cells/well approximately 24 h prior to treatment. mRNA-LNPs in PBS were diluted as necessary with PBS and added to the appropriate volume of media to obtain final treatment concentrations of 0, 0.03, 0.1, 0.3, 1 and 3 μg/mL mRNA concentrations. Treated cells analyzed for luciferase expression after 24 h. Cells were lysed using the Glo lysis buffer and treated with the luciferase reagent (both from Promega, Madison, WI) followed by a read-out using a luminometer.

### Example 1: LNP mRNA Systems Containing High Levels of Helper Lipids Exhibit Improved Gene Expression

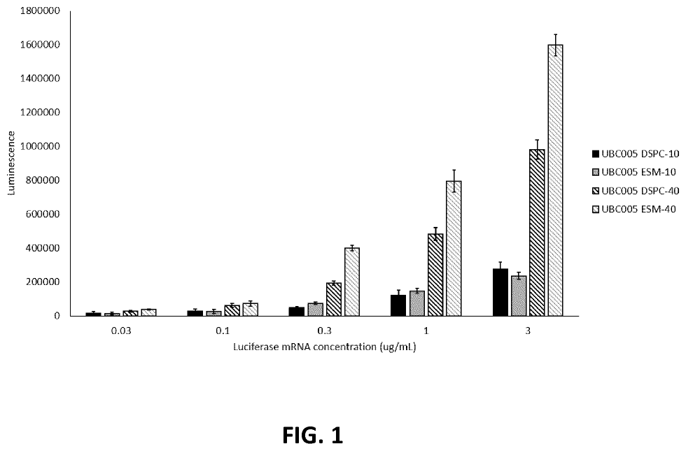

Current mRNA-LNP systems containing 10 mol % DSPC (the “Onpattro™-type” composition) can be effective agents for facilitating gene expression both in vitro and in the liver following i.v. administration. Here, the effect of increasing the proportions (from 10 mol % to 40 mol %) of the helper lipid, ESM, on the transfection potency of LNP containing luciferase mRNA (LNP Luc mRNA) in HuH7 (human derived hepatocarcinoma) cells was evaluated in vitro (see FIG. 1). The HuH7 cells were treated with LNPs containing different species and amounts of the helper lipid, holding the N/P ratio constant at 6:1. The N/P ratio is the cationic, ionizable lipid/nucleic acid charge ratio in the loaded LNP mRNA system. Optimized LNP mRNA formulations such as those used in vaccine applications use an N/P ratio of six.^{[2,7,18-20]}

The classical lipid composition used for Onpattro™ and LNP mRNA vaccines consists of ionizable lipid/DSPC/cholesterol/PEG-DMG in the molar proportions 50/10/38.5/1.5. As noted above, here we used the cationic, ionizable lipid, (6Z,9Z,26Z,29Z)-Pentatriaconta-6,9,26,29-tetraen-18-yl 4-(dimethylamino) butanoate (UBC005). The helper lipid content was increased to 40 mol % at the expense of both cholesterol and ionizable lipid, keeping the cholesterol-to-ionizable lipid molar ratio constant. The PEG-DMG content was maintained at 1.5 mol %. This corresponds to LNP lipid compositions ionizable, cationic lipid/helper lipid/cholesterol/PEG-lipid of 33/40/25.5/1.5 (mol/mol).

HuH7 cells were incubated with mRNA LNPs over a dose range of 0.03-3 μg mRNA/mL for 24 h and luciferase expression was then quantified by measuring luminescence as detailed in Methods. As shown in FIG. 1, the impact of increasing the helper lipid amount to 40 mol % (ESM) in formulations indicated that LNPs containing 40 mol % helper lipids were more potent than the LNPs containing 10 mol % helper lipid. For example, incubation (24 h) of HuH7 cells with LNP Luc mRNA formulations (3 μg mRNA/mL) containing 40 mol % ESM resulted in over a 7-fold increase in luciferase expression as compared to the 10 mol % ESM or DSPC Onpattro™-type formulation.

### Example 2: LNP mRNA Systems Containing 40 Mol % ESM Exhibit a Unique Morphology

The results of Example 1 demonstrate two advantageous features of the LNPs of the present disclosure. First, by increasing the proportions of non-toxic helper lipids in LNP mRNA systems, it is possible to not only maintain, but actually increase, transfection potency in vitro as compared to LNP mRNA with the “Onpattro™-type” lipid composition. Second, LNP mRNA systems containing 40 mol % ESM are significantly more potent transfection agents in vitro than LNP systems containing DSPC, DOPC, DOPE or DOPG. Thus, LNP mRNA systems containing 40 mol % ESM have the potential to not only enhance transfection potency in vivo but also to prolong circulation lifetimes and extend the range of tissues that the LNP can access and potentially transfect. Subsequent work was therefore focused on LNP mRNA systems containing high proportions of ESM.

The results of Example 1 compared LNP systems containing 40 mol % helper lipid with those containing 10 mol % DSPC. It was of interest to determine whether 40 mol % ESM was efficacious and also how the relative proportions of the other lipid components affect LNP properties.

In this regard, following the method of Example 1, the proportion of ESM was increased at the expense of both ionizable lipid and cholesterol, keeping the ratio of ionizable lipid to cholesterol constant. The resulting lipid compositions are summarized in Table 1 below.

The first variables to be characterized were the encapsulation efficiencies for Luc mRNA and LNP size and polydispersity (PDI). As noted in Table 1, high mRNA entrapment efficiencies of >95% were seen in LNPs formulated with 10 or 40 mol % ESM. The LNPs exhibited diameters ranging from 57-63 nm and had low PDI values.

TABLE 1
Influence of increasing ESM content at the expense of ionizable
lipid and cholesterol (keeping the ionizable lipid/cholesterol
ratio constant) on mRNA encapsulation efficiencies and LNP size

| | | | Sizing data | |
|---|---|---|---|---|
| | Lipid composition | Percent | Size | |
| Sample | (mol/mol) | encapsulation | (nm) | PDI |
| ESM10 | 50% ionizable, cationic | 96.88 | 57.7 | 0.063 |
| | lipid/38.5% chol/10% | | | |
| | ESM/1.5% PEG-DMG | | | |
| ESM40 | 33% ionizable, cationic | 98.13 | 62.22 | 0.092 |
| | lipid/25.5% chol/40% | | | |
| | ESM/1.5% PEG-DMG | | | |

Cryo-TEM studies reveal that increasing the proportions of ESM results in a transition from the commonly observed^{[21-23]} spherical “solid-core” structure seen for LNP containing 10 mol % ESM to a structure containing a core having an electron dense region and an aqueous portion surrounded by a bilayer for systems containing 40 mol % ESM content (FIG. 2A-B).

### Example 3: Suitable Method for In Vivo Analysis of GFP Gene Expression in the Liver, Spleen and/or Bone Marrow at 4 or 24 Hours Post-Injection

The following describes a suitable method for measuring in vivo expression of mRNA in the liver, spleen and/or bone marrow in a mouse model.

The mice were divided into groups of 2 and received intravenous (i.v.) injection of GFP mRNAs delivered LNPs based on Onpattro™-type, or a lipid nanoparticle mRNA composition in question, and PBS is used as a negative control. For biodistribution studies, LNPs entrapping GFP mRNA are labelled with 0.2 mol % DiD as fluorescent lipid marker. Injections are performed at 3 mg/kg mRNA dose and mice are sacrificed at 4 or 24 hours post injection (hpi). Mice are first anesthetized using a high dose of isofluorane followed by CO₂. Trans-cardiac perfusion is performed as follows: once the animals are unresponsive, a 5 cm medial incision is made through the abdominal wall, exposing the liver and heart. While the heart is still beating, a butterfly needle connected to a 30 mL syringe loaded with pre-warmed Hank's Balanced Salt Solution (HBSS, Gibco) is inserted into the left ventricle. Next, the liver is perfused with perfusion medium (HBSS, supplemented with 0.5 mM EDTA, Glucose 10 mM and HEPES 10 mM) at a rate of 3 mL/min for 10 min. Once liver swelling is observed, a cut is performed on the right atrium and perfusion is switched to digestion medium (DMEM, Gibco supplemented with 10% fetal bovine serum (FBS, Gibco) and 1% penicillin streptomycin (Gibco) and 0.8 mg/mL Collagenase Type IV, Worthington) at 3 mL/min for another 10 min. At the end of the perfusion of the entire system, as determined by organ blanching, the whole liver and spleen are dissected and transferred to 50 mL Falcon tubes containing 10 mL ice cold (4° C.) perfusion media and placed on ice.

Next, isolation of hepatocytes is performed following density gradient-based separation. Spleens and femurs are also harvested to isolate splenocytes and bone marrow cells. Briefly, the liver is transferred to a Petri dish containing digestion medium, minced under sterile conditions, and incubated for 20 min at 37° C. with occasional shaking of the plate. Cell suspensions are then filtered through a 40 μm mesh cell strainer to eliminate any undigested tissue remnants. Primary hepatocytes are separated from other liver residing cells by low-speed centrifugation at 500 rpm with no brake. The pellet containing mainly hepatocytes was collected, washed at 5000 rpm for 5 min and kept in 4° C. Femurs are centrifuged 10,000 g in a microcentrifuge for 10 seconds to collect the marrow that is resuspended in ammonium-chloride-potassium (ACK) lysis buffer for 1 min to deplete the red blood followed by washing with ice-cold PBS.

Phenotypic detection of hepatocytes is then performed using monoclonal antibodies to assess LNP delivery and mRNA expression. Cellular uptake and GFP expression is also detected in splenocytes and bone marrow cells immediately after isolation. Here, the spleen is dissected and placed into a 40 μm mesh cell and mashed through a cell strainer into a petri dish using a plunger end of a syringe. The suspended cells are transferred to a 15 mL Falcon™ tube and centrifuged at 1000 rpm for 5 minutes. The pellet is resuspended in 1 mL ACK lysis buffer (Invitrogen™) to lyse the red blood cells and aliquoted in FACS buffer. Cell aliquots are resuspended in 300 μl FACS staining buffer (FBS 2%, sodium azide 0.1% and ethylenediaminetetraacetic acid (EDTA 1 mM)) followed by staining with fluorescence tagged antibodies. Prior to staining, cells are first labeled with anti-mouse CD16/CD32 (mouse Fc blocker, Clone 2.4G2) (AntibodyLab™, Vancouver, Canada) to reduce background. Hepatocytes are detected following staining with primary mouse antibody detecting ASGR1 (8D7, Novus Biologicals) followed by goat polyclonal secondary antibody to mouse IgG2a labeled to PE-Cy7 (BioLegend™). Detection of hepatocytes, splenocytes and bone marrow cells is carried out using a LSRII flow cytometer and a FACSDiva™ software and analyzed by FlowJo™ following acquisition of 1,000,000 events after gating on viable cell populations. LNP-mRNA delivery or transfection efficacy is assessed based on the relative mean fluorescence intensity of DiD or GFP positive cells, respectively, measured on histograms obtained from gated cell populations.

Statistical analyses are performed using a two-tailed Student's t-test, where groups are compared. The type (paired or two-sample equal variance-homoscedastic), is determined based on the variation of the standard deviation of two populations. P<0.05 is accepted as statistically significant (*P<0.05).

### Example 5: Results of In Vivo Analysis of GFP Gene Expression in the Liver, Spleen and/or Bone Marrow at 4 Hours Post-Injection

This example summarizes the results of in vivo studies examining GFP gene expression in the liver, spleen and/or bone marrow at 4 hours post-injection of LNPs with 10 mol % sphingomyelin in an Onpattro™-type formulation (using UBC005) or LNPs with 40 mol % sphingomyelin following the procedures of Example 4. The cationic, ionizable lipid used on the formulations was (6Z,9Z,26Z,29Z)-Pentatriaconta-6,9,26,29-tetraen-18-yl 4-(dimethylamino) butanoate.

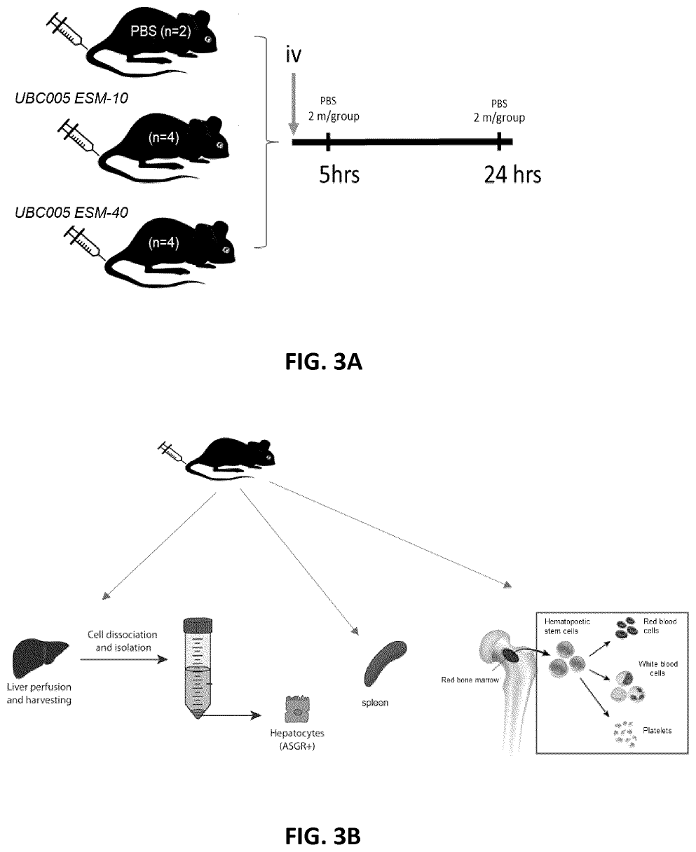

The formulations tested are set out in Table 2 below. FIGS. 3A and 3B summarize the experimental design. The animal model used in the studies was C57B16 mice. Gene expression was determined at four hours post injection.

TABLE 2
Formulations used in the in vivo analysis of GFP gene expression in
the liver, spleen and/or bone marrow at 4 hours post-injection (hpi)

| Sample | Lipid composition |
|---|---|
| PBS | Phosphate buffered saline control |
| UBC005 | 50% ionizable, cationic lipid/38.5% |
| (ESM-10) | chol/10% ESM/1.5% PEG-DMG |
| lcLNP | 33% ionizable, cationic lipid/25.5% |
| (ESM-40) | chol/40% ESM/1.5% PEG-DMG |

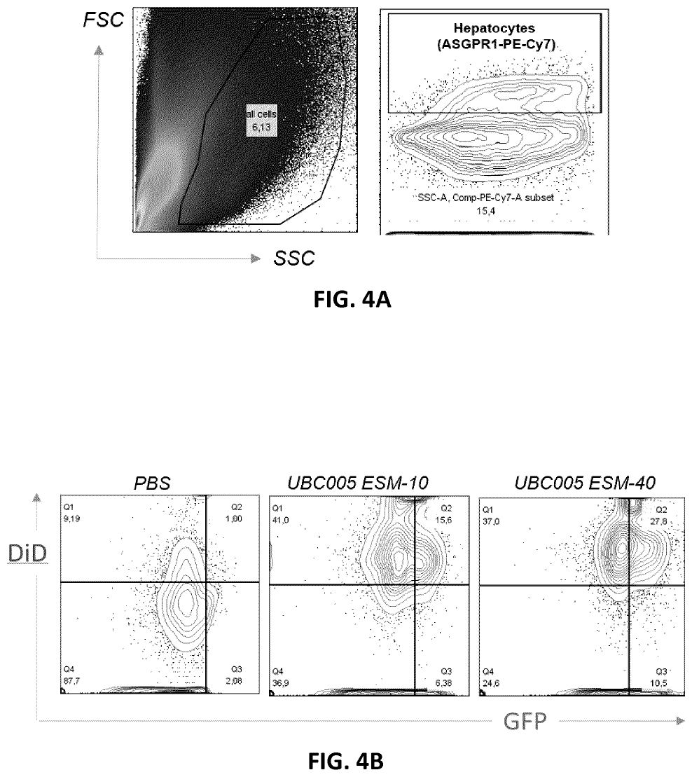

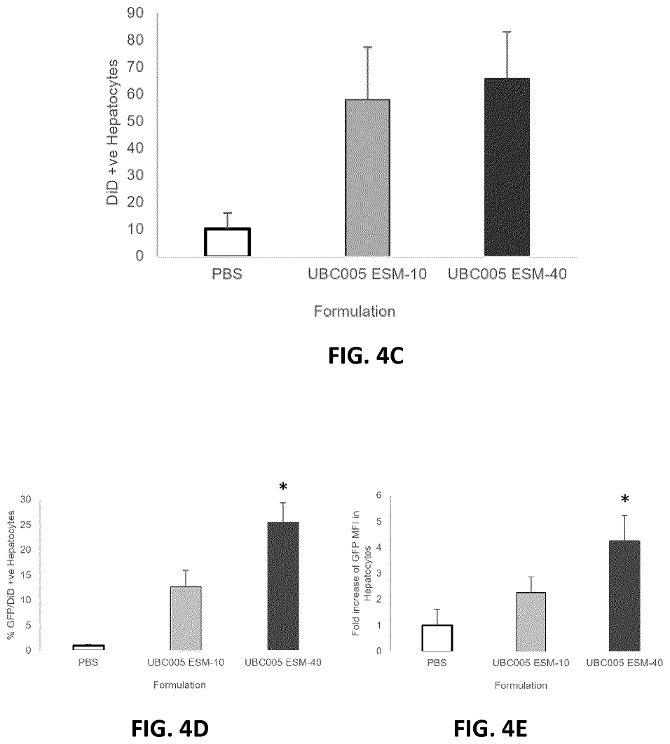

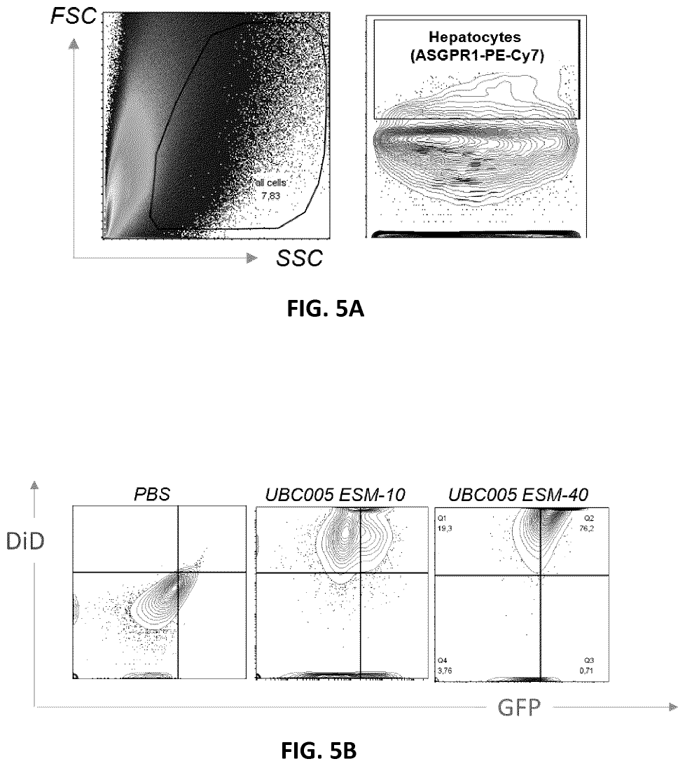

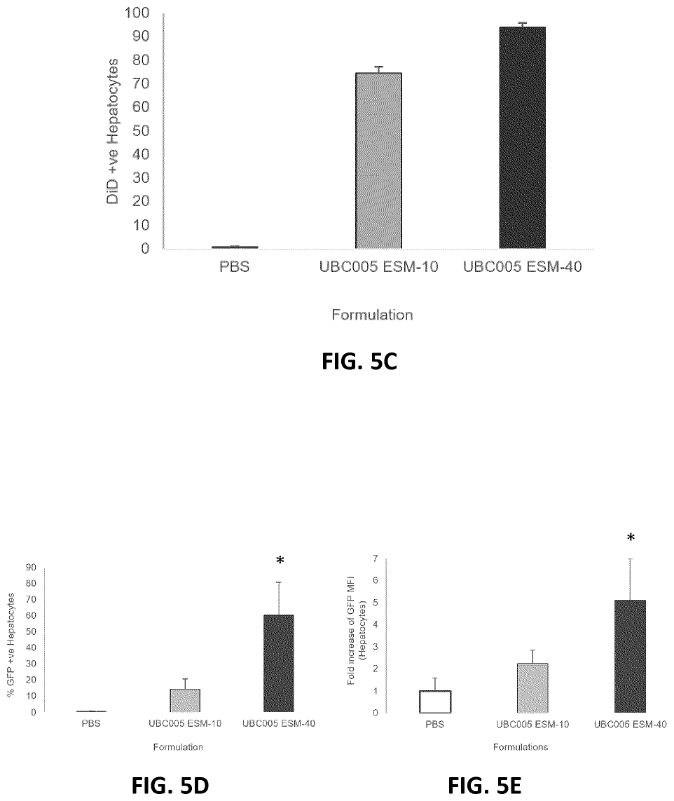

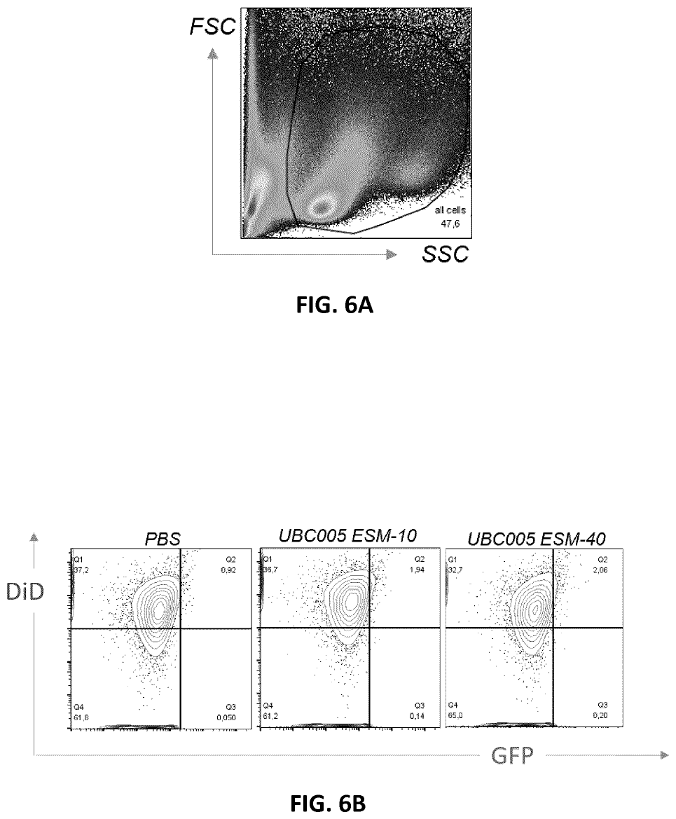

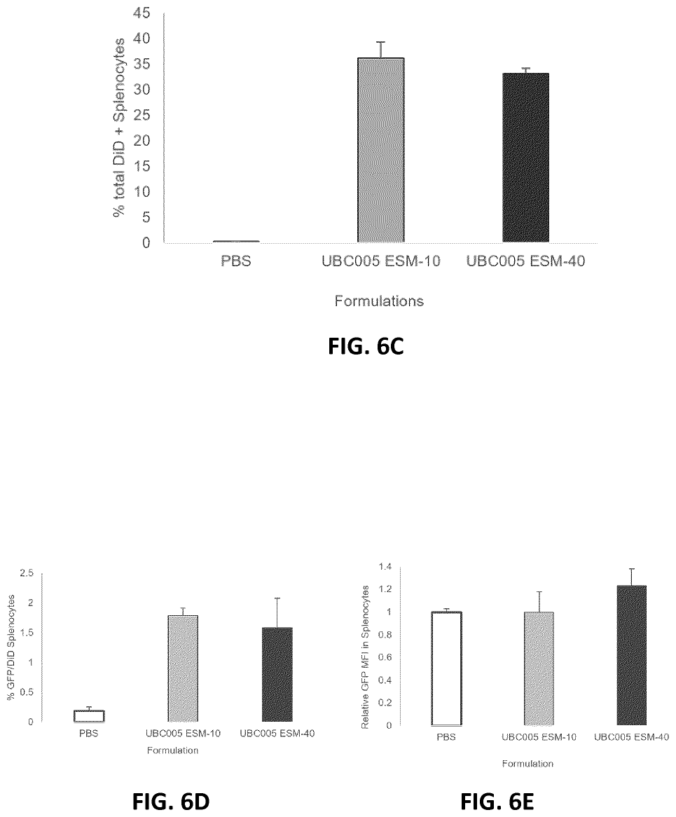

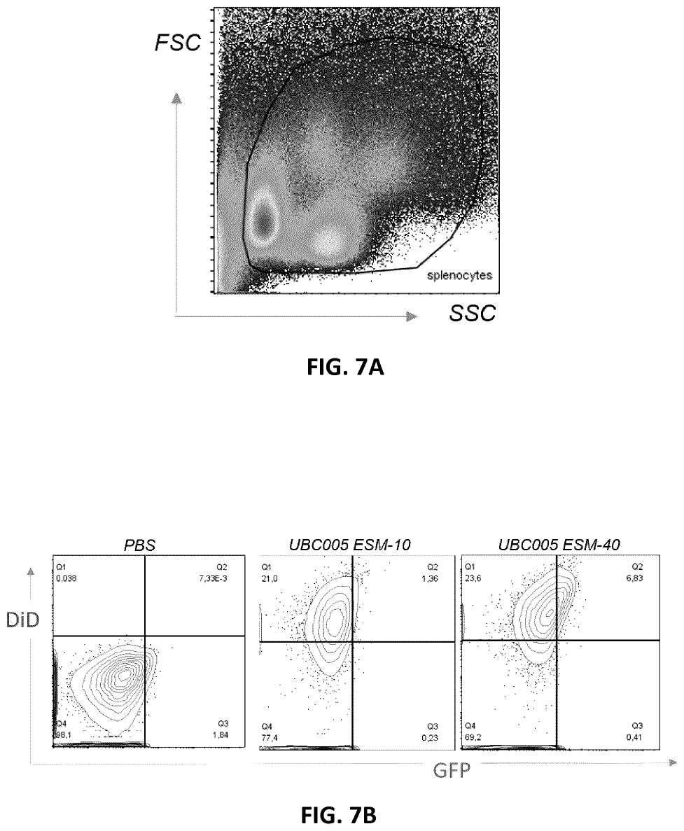

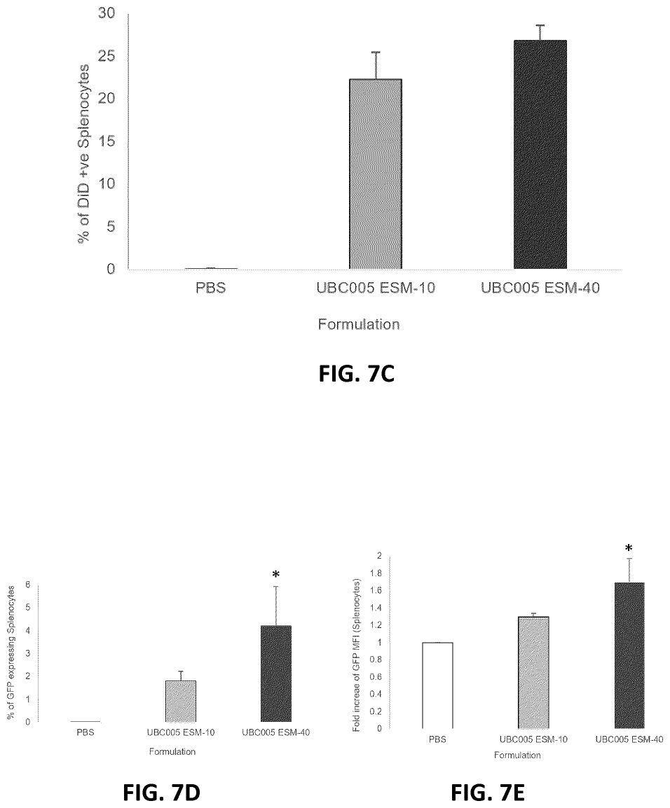

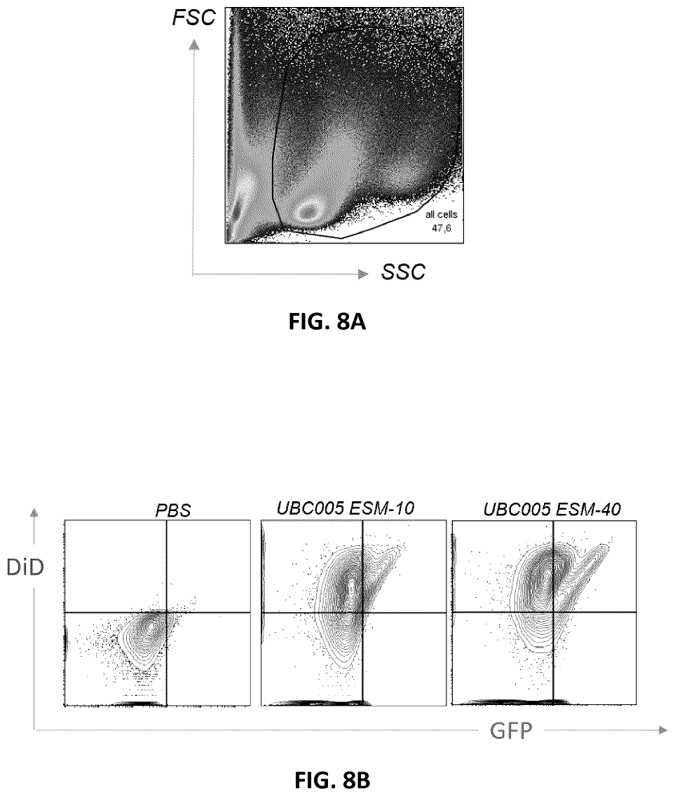

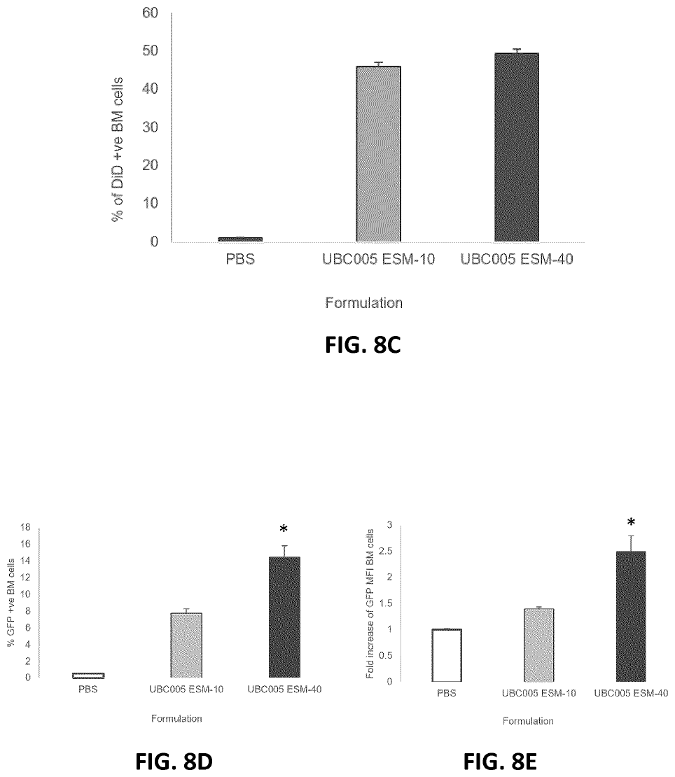

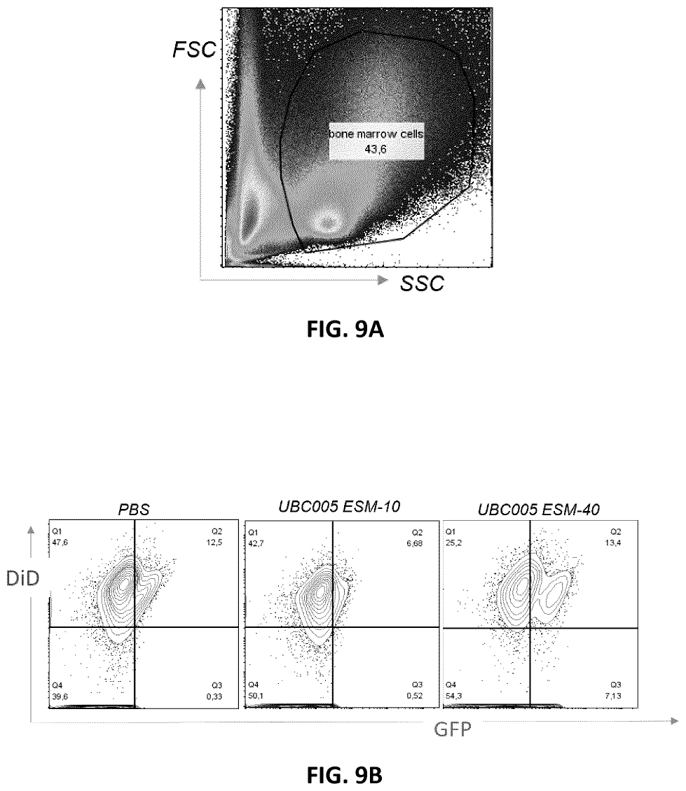

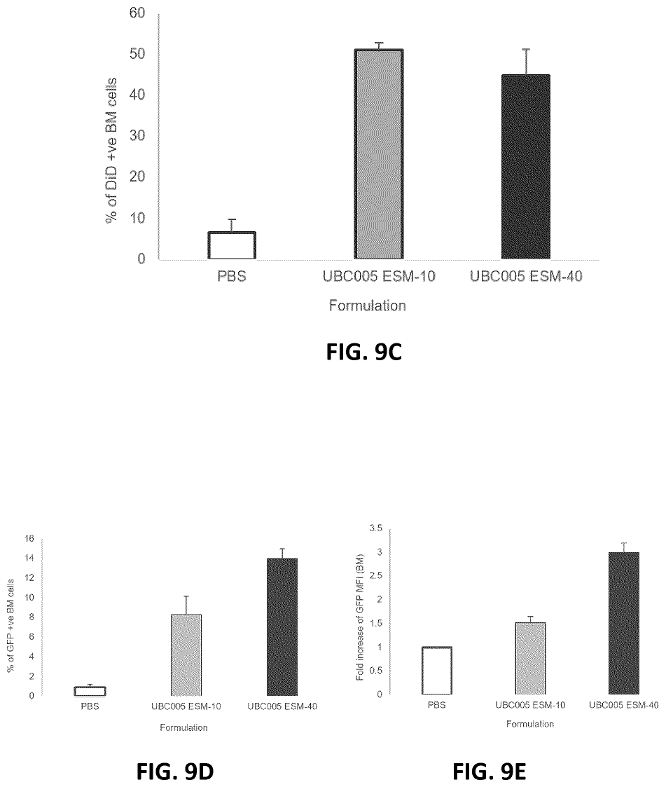

The results are shown in FIGS. 4-9. Surprisingly, the fold-increase GFP expression of the inventive compositions (lcLNP ESM-40) relative to the Onpattro™-type formulations was at least 2-fold in cells of a least one of the liver, spleen or bone marrow. The data shows that the four-component lipid nanoparticles having elevated levels of sphingomyelin (lcLNPs (ESM-40)) are an effective delivery tool to induce expression of GFP in hepatocytes, splenocytes and bone marrow cells relative to the UBC005 (ESM10) formulation (see Table 2 above).

The results also show that the lcLNP (ESM-40) yields higher GFP expression in hepatocytes>bone marrow (BM) cells>spleen at later time points. These results suggest that the translation efficiency is more prominent in hepatocytes.

### Example 6: Results of In Vivo Analysis of Luciferase Gene Expression in Fifteen Organs at 5 and 24 Hours Post-Injection

Formulations comprising mRNA encoding luciferase and having the lipid composition of Table 3 were intraperitoneally administrated to Female CD1 mice intravenously (2 mg/kg) and D-luciferin (150 mg/kg).

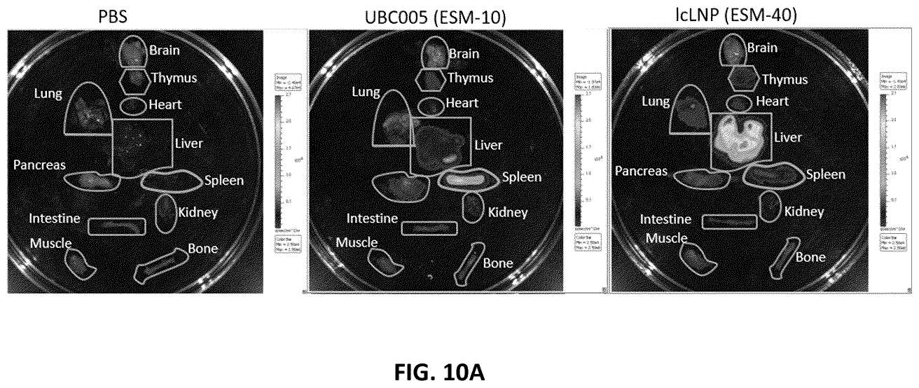

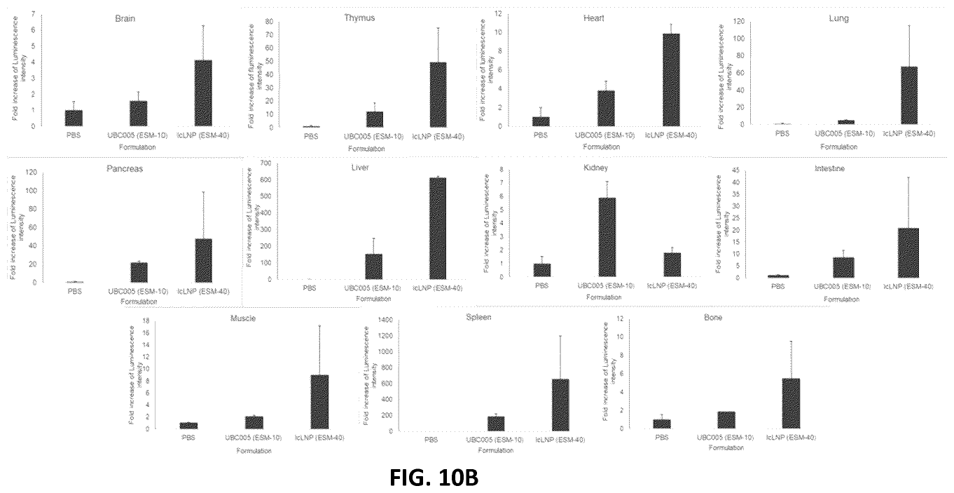

TABLE 3
Formulations examined in FIGS. 10-12

| Sample | Lipid composition |
|---|---|
| PBS | Phosphate buffered saline control |
| UBC005 | 50% ionizable, cationic lipid/38.5% |
| (DSPC-10) | chol/10% DSPC/1.5% PEG-DMG |
| lcLNP | 20% ionizable, cationic lipid/38.5% |
| (ESM-40) | chol/40% ESM/1.5% PEG-DMG |

FIG. 10A shows the ex vivo bioluminescence images of 15 organs (brain, thymus, heart, lungs, liver, spleen, pancreas, intestine, kidney, muscle, and bone) at 5 hours post-injection using an IVIS Spectrum imaging system. As can be visualized, the mRNA-lcLNP comprising elevated sphingomyelin (40 mol %) exhibited stronger bioluminescence signals relative to mRNA-LNP having only 10 mol % DSPC (UBC005 (DSPC-10)).

At 5 hours post injection, the lcLNP (ESM-40) formulation having high sphingomyelin content (40 mol %) exhibited improved tissue delivery to the majority of organs examined (brain, thymus, heart, lung, pancreas, liver, intestine, muscle, spleen and bone) relative to the UBC005 (DSPC-10) formulation having only 10 mol % DSPC (FIG. 10B) as measured using photon flux (photons/second) using the Living IMAGE™ Software.

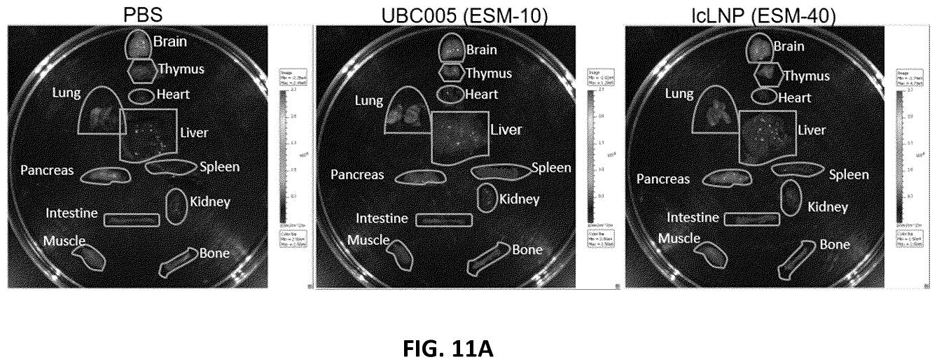

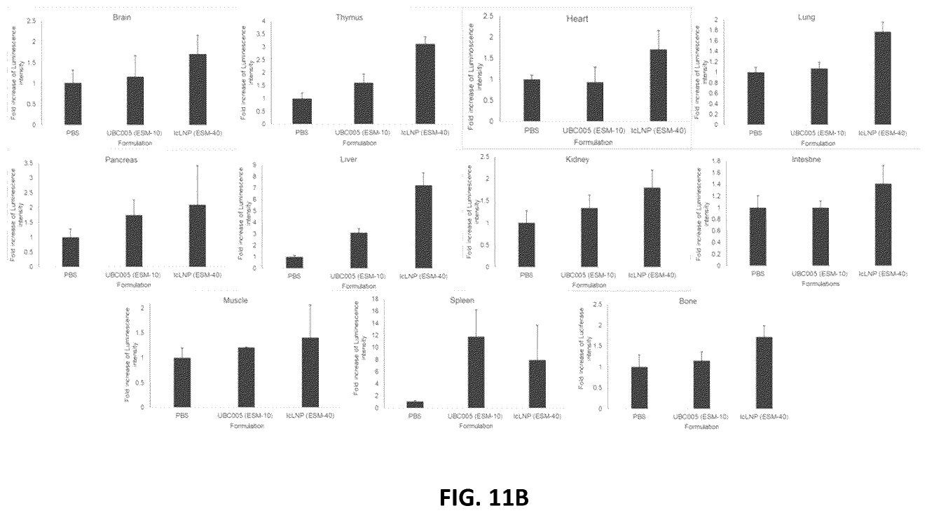

Likewise, at 24 hours post injection, the lcLNP (ESM-40) formulation having high sphingomyelin content (40 mol %) exhibited improved tissue delivery to the majority of organs examined (brain, thymus, heart, lung, pancreas, liver, kidney, intestine, muscle and bone) relative to the UBC005 (DSPC-10) formulation having only 10 mol % DSPC (FIG. 11B) as measured using photon flux (photons/second) using the Living IMAGE™ Software.

Thus, surprisingly, at both time points examined, delivery of encapsulated mRNA in the majority of tissues examined (as measured by bioluminescence) was increased for the LNPs having elevated levels of sphingomyelin lcLNP (ESM-40) over the formulation having only 10 mol % helper lipid (DSPC-10).

### Example 7: Results of mRNA Serum Stability Studies Comparing Elevated Sphingomyelin mRNA-LNPs to mRNA-LNPs Having 10 Mol % DSPC

The ability of mRNA in LNPs having elevated levels of sphingomyelin to withstand degradation in vitro when incubated in serum relative to a formulation having low helper lipid content was next investigated. The LNPs tested were four-component systems (ionizable lipid/cholesterol/helper lipid/PEG-lipid) that contained 40 mol % sphingomyelin or 10 mol % DSPC. The compositions tested included those set out in Table 3 above (ESM40 and UBC005 (DSPC-10)) and unencapsulated mRNA (“naked mRNA”). The ESM40, UBC005 (DSPC-10) and naked mRNA samples were incubated in 50% fetal bovine serum (FBS) for 0, 2, 4 and 24 hours. The mRNA-LNP comprising 10 mol % DSPC ((UBC005 (DSPC-10)) and naked mRNA were also incubated in phosphate buffered saline (PBS) as a positive control. For LNP samples, the mRNA was extracted from the LNP-mRNA formulations at the time points indicated and run on the denaturing agarose gel. Extracted RNAs were also loaded into a RNA Chip kit and mRNA integrity was determined by automated electrophoresis using Agilent 2100 Bioanalyzer. Electropherograms of the mRNA samples were generated and used to determine mRNA protection in the formulation following incubation in PBS or serum as indicated. Visual inspection of the electropherogram (FIG. 12C) demonstrates that at 0 time point, all extracts exhibit two typical mRNA peaks (lanes A-E). However, with increasing the incubation time in serum, the mRNA profile undergoes changes 2 hrs following incubation in serum (Lane B) suggesting some degradation of mRNA transcripts in the UBC005 (DSPC-10) mRNA formulation. The mRNA formulated with lcLNP (ESM-40), on the other hand, demonstrated a slower degradation exhibited by the presence of the predominant mRNA peak (Lane C) until 4 hours of incubation in serum. Twenty-four hours following incubation in serum, mRNA profile demonstrated almost complete degradation in both UBC005 (DSPC-10) and lcLNP (ESM-40), similar to naked mRNA (Lane A). The electropherogram demonstrated that mRNA remained intact during the course of 24 hours for both formulations when incubated in PBS (Lane D and A). Taken together, the data demonstrates a slower degradation process of mRNA when formulated with lcLNP (ESM-40) composition. Furthermore, by agarose gel, less degradation of mRNA in the LNPs having elevated levels of sphingomyelin (40 mol %) relative to the LNPs having only 10 mol % DSPC. As can be seen in the agarose gel of FIG. 12B, bands of RNA can be visualized in the LNP having 40 mol % ESM (ESM40), while only a band at 0 hours was visualized for mRNA-LNP comprising UBC005 (DSPC-10). The normalized absorption ratio data in FIG. 12A also suggests that the 40 mol % ESM formulations had lower levels of mRNA degradation relative to the 10 mol % DSPC formulations as determined by normalized absorption of each band at each time point tested.

Although the invention has been described and illustrated with reference to the foregoing examples, it will be apparent that a variety of modifications and changes may be made without departing from the invention.

### REFERENCES

[1] K. A. Whitehead, R. Langer, D. G. Anderson, Nat Rev Drug Discov 2009, 8, 129.
[2] M. A. Oberli, A. M. Reichmuth, J. R. Dorkin, M. J. Mitchell, O. S. Fenton, A. Jaklenec, D. G. Anderson, R. Langer, D. Blankschtein, Nano Lett 2017, 17, 1326.
[3] A. Akinc, M. A. Maier, M. Manoharan, K. Fitzgerald, M. Jayaraman, S. Barros, S. Ansell, X. Du, M. J. Hope, T. D. Madden, et al., Nat Nanotechnol 2019, 14, 1084.
[4] T. M. Allen, P. R. Cullis, Advanced Drug Delivery Reviews 2013, 65, 36.
[5] N. Pardi, M. J. Hogan, R. S. Pelc, H. Muramatsu, H. Andersen, C. R. DeMaso, K. A. Dowd, L. L. Sutherland, R. M. Scearce, R. Parks, et al., Nature 2017, 543, 248.
[6] K. S. Park, X. Sun, M. E. Aikins, J. J. Moon, Adv Drug Deliv Rev 2021, 169, 137.
[7] M. D. Buschmann, M. J. Carrasco, S. Alishetty, M. Paige, M. G. Alameh, D. Weissman, Vaccines 2021, 9, 65.
[8] “Stabilized plasmid-lipid particles: construction and characterization|Gene Therapy,” can be found under https://www.nature.com/articles/3300821, n.d.
[9] A. Gabizon, H. Shmeeda, Y. Barenholz, Clinical pharmacokinetics 2003, 42, 419.
[10] T. M. Allen, C. Hansen, F. Martin, C. Redemann, A. Yau-Young, Biochimica et Biophysica Acta (BBA)—Biomembranes 1991, 1066, 29.
[11] S. C. Semple, A. Akinc, J. Chen, A. P. Sandhu, B. L. Mui, C. K. Cho, D. W. Y. Sah, D. Stebbing, E. J. Crosley, E. Yaworski, et al., Nat Biotechnol 2010, 28, 172.
[12] J. H. Felgner, R. Kumar, C. N. Sridhar, C. J. Wheeler, Y. J. Tsai, R. Border, P. Ramsey, M. Martin, P. L. Felgner, J Biol Chem 1994, 269, 2550.
[13] S. A. L. Audouy, L. F. M. H. de Leij, D. Hoekstra, G. Molema, Pharm Res 2002, 19, 1599.
[14] M. S. Webb, T. O. Harasym, D. Masin, M. B. Bally, L. D. Mayer, Br J Cancer 1995, 72, 896.
[15] T. M. Allen, C. Hansen, J. Rutledge, Biochim Biophys Acta 1989, 981, 27.
[16] N. M. Belliveau, J. Huft, P. J. Lin, S. Chen, A. K. Leung, T. J. Leaver, A. W. Wild, J. B. Lee, R. J. Taylor, Y. K. Tam, et al., Mol Ther Nucleic Acids 2012, 1, e37.
[17] A. K. K. Leung, Y. Y. C. Tam, S. Chen, I. M. Hafez, P. R. Cullis, J Phys Chem B 2015, 119, 8698.
[18] K. J. Hassett, K. E. Benenato, E. Jacquinet, A. Lee, A. Woods, O. Yuzhakov, S. Himansu, J. Deterling, B. M. Geilich, T. Ketova, et al., Molecular Therapy—Nucleic Acids 2019, 15, 1.
[19] N. Pardi, S. Tuyishime, H. Muramatsu, K. Kariko, B. L. Mui, Y. K. Tam, T. D. Madden, M. J. Hope, D. Weissman, J Control Release 2015, 217, 345.
[20] K. J. Kauffman, J. R. Dorkin, J. H. Yang, M. W. Heartlein, F. DeRosa, F. F. Mir, O. S. Fenton, D. G. Anderson, Nano Lett. 2015, 15, 7300.
[21] A. K. K. Leung, Y. Y. C. Tam, P. R. Cullis, Adv Genet 2014, 88, 71.
[22] R. Crawford, B. Dogdas, E. Keough, R. M. Haas, W. Wepukhulu, S. Krotzer, P. A. Burke, L. Sepp-Lorenzino, A. Bagchi, B. J. Howell, Int J Pharm 2011, 403, 237.
[23] Y. Eygeris, S. Patel, A. Jozic, G. Sahay, Nano Lett. 2020, 20, 4543.
[24] P. Tam, M. Monck, D. Lee, O. Ludkovski, E. C. Leng, K. Clow, H. Stark, P. Scherrer, R. W. Graham, P. R. Cullis, Gene Ther 2000, 7, 1867.
[25] M. Jayaraman, S. M. Ansell, B. L. Mui, Y. K. Tam, J. Chen, X. Du, D. Butler, L. Eltepu, S. Matsuda, J. K. Narayanannair, et al., Angew Chem Int Ed Engl 2012, 51, 8529.
[26] C. A. Alabi, K. T. Love, G. Sahay, H. Yin, K. M. Luly, R. Langer, D. G. Anderson, PNAS 2013, DOI 10.1073/pnas. 1306529110.
[27] M. Ordobadi, Lipid Nanoparticles for Delivery of Bioactive Molecules, University of British Columbia, 2019.
[28] S. Chira, C. S. Jackson, I. Oprea, F. Ozturk, M. S. Pepper, I. Diaconu, C. Braicu, L.-Z. Raduly, G. A. Calin, I. Berindan-Neagoe, Oncotarget 2015, 6, 30675.
[29] J. A. Kulkarni, M. M. Darjuan, J. E. Mercer, S. Chen, R. van der Meel, J. L. Thewalt, Y. Y. C. Tam, P. R. Cullis, ACS Nano 2018, 12, 4787.
[30] J. A. Kulkarni, D. Witzigmann, J. Leung, R. van der Meel, J. Zaifman, M. M. Darjuan, H. M. Grisch-Chan, B. Thony, Y. Y. C. Tam, P. R. Cullis, Nanoscale 2019, 11, 9023.

1. A lipid nanoparticle comprising encapsulated mRNA and 30 to 60 mol % of a sphingolipid, and at least one of a sterol and a hydrophilic polymer-lipid conjugate, the lipid nanoparticle comprising a core having an electron dense region and an aqueous portion surrounded at least partially by a lipid layer comprising at least a bilayer and the lipid nanoparticle exhibiting at least a 2-fold increase in gene expression in the liver, spleen and/or bone marrow at 4 or 24 hours post-injection as compared to a lipid nanoparticle encapsulating the mRNA with a formulation of ionizable, cationic lipid/DSPC/cholesterol/PEG-lipid or ionizable, cationic lipid/egg sphingolipid/cholesterol/PEG-lipid at 50/10/38.5/1.5, mol:mol, wherein the gene expression is measured in an animal model by detection of green fluorescent protein (GFP) or luciferase.

2. A lipid nanoparticle for hepatic or extrahepatic delivery of mRNA, the lipid nanoparticle comprising:
(i) encapsulated mRNA;
(ii) a sphingolipid content of from 30 mol % to 60 mol % of total lipid present in the lipid nanoparticle;
(iii) a cationic lipid content of from 5 mol % to 50 mol % of the total lipid;
(iv) a sterol selected from cholesterol or a derivative thereof; and
(v) a hydrophilic polymer-lipid conjugate that is present at 0.5 mol % to 5 mol %, or at 0.5 mol % to 3 mol % of the total lipid,
the lipid nanoparticle having a core comprising an electron dense region and an aqueous portion surrounded at least partially by a lipid layer comprising at least a bilayer.

3. The lipid nanoparticle of claim 1, wherein the sphingolipid content is between 30 mol % and 50 mol % or between 30 mol % and 50 mol %.

4. The lipid nanoparticle of claim 2, wherein the sphingolipid content is between 35 mol % and 60 mol % or between 30 mol % and 50 mol %.

5. The lipid nanoparticle of claim 1, wherein the electron dense region is denser than the aqueous portion as visualized by cryo-EM microscopy.

6. The lipid nanoparticle of claim 5, wherein the lipid nanoparticle is part of a preparation of lipid nanoparticles, and wherein the electron dense region of at least 20% of the lipid nanoparticles are either (i) enveloped by the aqueous portion, or (ii) is partially surrounded by the aqueous portion and wherein a portion of a periphery of the electron dense region is contiguous with the lipid layer, as visualized by cryo-EM microscopy.

7. The lipid nanoparticle of claim 1, wherein at least a portion of the mRNA is encapsulated in the electron dense region or the lipid layer.

8. The lipid nanoparticle of claim 1, wherein the cationic lipid is an amino lipid.

9. The lipid nanoparticle of claim 1, wherein the cationic lipid has a pKa of between 6.0 and 7.2.

10. The lipid nanoparticle of claim 1, wherein the hydrophilic polymer-lipid conjugate is a polyethyleneglycol-lipid conjugate.

11. The lipid nanoparticle of claim 1, wherein the sterol is present at from 15 mol % to 50 mol % based on the total lipid present in the lipid nanoparticle.

12. The lipid nanoparticle of claim 1, wherein the sterol is present at from 18 mol % to 45 mol % based on the total lipid present in the lipid nanoparticle.

13. The lipid nanoparticle of claim 1, wherein the sphingolipid is sphingomyelin.

14. The lipid nanoparticle of claim 1, wherein the mRNA stability of the lipid nanoparticle is improved relative to the formulation of ionizable, cationic lipid/DSPC/cholesterolPEG-lipid at 50/10/38.5/1.5, mol:mol as measured by quantifying degradation in an in vito assay by determining band intensity using a denaturing agarose gel after incubation of the lipid nanoparticle with fetal bovine serum for 2, 4 or 24 hours, wherein the mRNA stability improvement is measured by determining a normalized absorption ratio for peaks at λ 260 nm and λ 280 nm (λ 260 nm/λ 280 nm) for the lipid nanoparticle, and wherein the normalized absorption ratio is least 0.5, 1.0, 1.5 or 2% greater than that of the cationic lipid/DSPC/cholesterol/PEG-lipid at 50/10/38.5/1.5, mol:mol at any one of the 2, 4 or 24 hours.

15. A method for in vivo delivery of mRNA to a hepatic or extrahepatic tissue or an organ to treat or prevent a disease or disorder in a mammalian subject, the method comprising:
administering to the mammalian subject a lipid nanoparticle of claim 1.

16. The method of claim 15, wherein the lipid nanoparticle is for delivery to spleen, bone marrow or liver.

17. The method of claim 16, wherein the lipid nanoparticle is for delivery to the liver.

18. The method of claim 15, wherein the disease or disorder is a viral infection.

19. The method of claim 15, wherein the disease or disorder is cancer.

20-24. (canceled)

25. The lipid nanoparticle of claim 2, wherein the electron dense region is denser than the aqueous portion as visualized by cryo-EM microscopy.
